(19) United States
(12) Patent Application Publication
(10) Pub. No.: US 2018/0110497 A1
(43) Pub. Date: Apr. 26, 2018

(54) CUSTOMIZED HANDLE FOR ULTRASOUND PROBE

(71) Applicant: General Electric Company, Schenectady, NY (US)

(72) Inventors: Jimmie Autrey Beacham, West Allis, WI (US); Kevin Rooney, Pewaukee, WI (US)

(21) Appl. No.: 15/334,232

(22) Filed: Oct. 25, 2016

(51) Int. Cl. A61B 8/00; G06F 17/50; B29C 65/00

(52) U.S. Cl. CPC A61B 8/4455; G06F 17/50; B33Y 10/00; G06F 2217/12; B29C 65/00

(57) ABSTRACT

A method of manufacturing an ultrasound probe comprises customizing a fit of the ultrasound probe to an operator's hand, including, generating a three-dimensional (3D) model of the operator's hand, digitizing the 3D model of the operator's hand, including obtaining a set of manual attributes, and forming a manually grasped surface of the ultrasound probe based on the digitized 3D model, and coupling the manually grasped surface to the ultrasound probe. In this way, operator hand strain while conducting ultrasound exams can be reduced.

### FIELD

Embodiments of the subject matter disclosed herein relate to an ultrasound probe having a customized handle and methods of manufacturing thereof.

### BACKGROUND

Ultrasound probes are devices that send and receive ultrasonic sound waves, and are used extensively in the healthcare industry for imaging of internal organs. Ultrasound probes come in many shapes and sizes. The size and shape of an ultrasound probe may determine its field of view, and the frequency of emitted ultrasound waves determines how deep the sound waves penetrate and the resolution of the image. In addition, an ultrasound probe may be selected for a particular clinical application based on its shape, size, and scanning characteristics.

For example, Szabo et. al. (J. Ultrasound Med., 2013; 32: 573-582) discuss grouping transducer probes based on their physical dimensions, footprint contact area, shape, and imaging format, and then develop a systematic method for selecting a transducer probe based on these criteria for a particular clinical application. Imaging format criteria include access to and coverage of the region of interest, maximum scan depth and image extent, and coverage of essential diagnostic modes required to optimize a patient's diagnosis.

The inventors herein have recognized various issues with the above approach. Namely, the selection, design, and shape of conventional ultrasound probes fail to account for ergonomic considerations including the size, shape and features of an operator's hand. In particular, because conventional probe handles do not conform to an operator's hand or grip, an operator's hand may easily fatigue and become strained while performing ultrasound scans for a patient due to prolonged repetitive motions. Furthermore, ultrasound probes are typically shared amongst several operators, which can be unsanitary. Further still, when a handle of an ultrasound probe cracks or fails, the entire ultrasound probe must be replaced, which increases clinical operating costs.

### BRIEF DESCRIPTION

In one embodiment, the issues described above may be at least partially addressed by a method of manufacturing an ultrasound probe, comprising: customizing the fit of the ultrasound probe to a operator's hand, including, generating a three-dimensional (3D) model of the operator's hand, digitizing the 3D model of the operator's hand, including obtaining a set of manual attributes, and forming a manually grasped surface of the ultrasound probe based on the digitized 3D model; and coupling the manually grasped surface to the ultrasound probe.

In another embodiment, a method of manufacturing an ultrasound probe comprises: forming a manually grasped surface of the ultrasound probe corresponding to a model of a grasping hand, wherein the model includes a set of manual attributes that identify the grasping hand, and the manually grasped surface comprises a negative surface conforming to a positive surface including the grasping hand, and attaching the manually grasped surface to the ultrasound probe.

In another embodiment, an ultrasound probe comprises: a housing, including a manually grasped surface corresponding to a model of a grasping hand, wherein the model includes a set of manual attributes that identify the grasping hand, and the manually grasped surface comprises a negative surface conforming to a positive surface including the grasping hand; probe electronics, including an ultrasound probe transducer, positioned inside the housing; and a lens conductively coupled to the probe electronics, positioned at a periphery of the housing, and through which ultrasound radiation is transmitted and received through the housing.

In this way, a technical effect is achieved where ultrasound probes may be designed to be (fully or partially) customizable to the size and shape of a operator's hand, thereby reducing injuries and discomfort due to ergonomic strain and chronic fatigue of the operator's hand and wrist. Furthermore, existing ultrasound probes can be retrofitted with a custom-fit or partially custom-fit ultrasound probe handle, thereby reducing replacement costs. Further still, the custom-fit handles may be removably attached, thereby facilitating repair and reducing replacement costs. Further still, custom-fitting ultrasound probes for each operator can improve hygiene and reduce contamination issues resulting from common ultrasound probes shared amongst several operators. Further still, custom-fitting the ultrasound probe handle to a operator's hand may increase interior free volume within the ultrasound probe, allowing for additional heat dissipation devices to be housed within the ultrasound probe, and thereby reducing degradation and prolonging the useable life of the probe. Further still, custom-fitting ultrasound probes can encourage standardization of hand and wrist posture while grasping ultrasound probes across operators, which can reduce operator to operator variation and increase the reliability of ultrasound imaging.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIGS. 1 and 11 are schematics showing perspective views of various conventional ultrasound probes.

FIG. 2 is a schematic illustrating neutral and non-neutral hand postures.

FIGS. 3 and 4 are schematics showing perspective views of various operators grasping conventional ultrasound probes.

FIGS. 5 and 12 are schematics illustrating various manual attributes.

FIGS. 6, 7, and 10 are schematics showing methods of generating and digitizing a model of a operator's hand.

FIG. 8 is a schematic showing custom-fit ultrasound probes.

FIG. 9 is an example flow chart for a method of manufacturing the ultrasound probes of FIG. 8.

### DETAILED DESCRIPTION

The following description relates to various embodiments of an ultrasound probe and methods of manufacturing an ultrasound probe.

In one embodiment, a method of manufacturing an ultrasound probe comprises: customizing the fit of the ultrasound probe to a operator's hand, including, generating a three-dimensional (3D) model of the operator's hand, digitizing the 3D model of the operator's hand, including obtaining a set of manual attributes, and forming a manually grasped surface of the ultrasound probe based on the digitized 3D model; and coupling the manually grasped surface to the ultrasound probe.

Ultrasound imaging (sonography) uses high-frequency sound waves to view inside of a patient's body. Because ultrasound images are captured in real-time, they can also show movement of the body's internal organs as well as blood flowing through the blood vessels. During an ultrasound exam, an ultrasound probe (transducer) is placed directly in contact with a patient's skin or inside a patient's body opening. A thin layer of gel may be applied to the skin underneath the ultrasound probe to aid in directing ultrasound waves from the probe through the gel into the body. The ultrasound probe can be moved along the surface of the body (or within a body cavity) and angled or oriented to obtain various perspectives inside the body. In many clinical applications, such as maternal abdomen ultrasound exams, it is common for the operator to continually grasp the ultrasound probe for prolonged periods of time, which can give rise to fatigue and strain of the operator's hand and wrist.

An ultrasound imaging system can include: the ultrasound probe with controls for changing the amplitude, duration, and frequency of the ultrasound signals emitted from the probe; and a computer that performs calculations and provides \power source for itself and the transducer, displays an image based on the ultrasound data processed by the computer, receives input from the operator and obtains measurements from the display. Compared to other common methods of medical imaging, ultrasound has several advantages: it provides images in real-time; it is portable and can be brought to the bedside; it is substantially lower in cost; and it does not use harmful ionizing radiation.

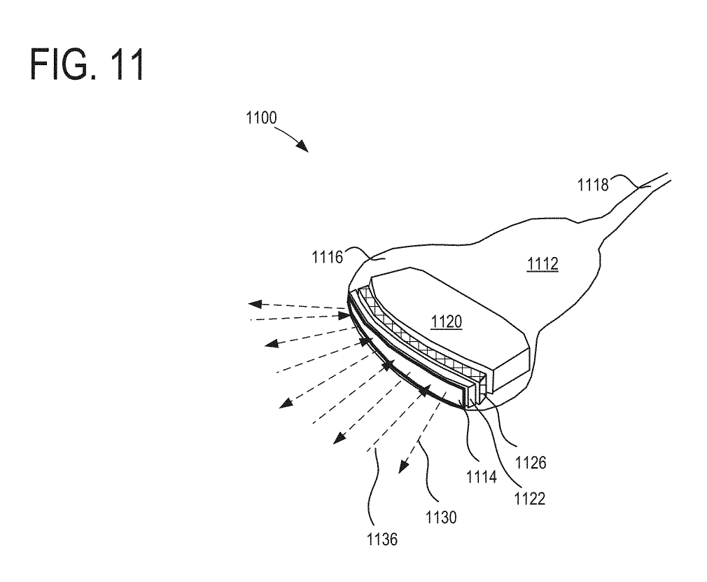

Turning now to FIG. 11, it illustrates a conventional ultrasound probe 1100 including a lens 1114, a handle 1112, a probe tip 1116, cabling 1118, an acoustic matching layer 1122, a piezoelectric element 1126, and a backing material 1120. Upon application of voltage, the piezoelectric element 1126 repeatedly expands and contracts, thereby generating ultrasonic waves 1130 which are directed outward from the probe through the lens 1124 towards an object being imaged (e.g., a patient's body). Conversely, reflected ultrasonic radiation from the subject incident at the piezoelectric element 1126 via the lens 1114, generates a voltage at the piezoelectric element 1126, which can be interpreted by a computer processor coupled to the probe through cabling 1118. Backing material 1120 is positioned posteriorly to the piezoelectric element 1126 relative to the lens 1114 to dampen the ultrasonic vibrations arising therein, which aids in improving image resolution by giving rise to ultrasonic waves with shorter pulse lengths. To improve transmission and penetration of the ultrasonic waves generated from the probe into an object being imaged (and to reduce reflection or scattering of ultrasonic waves at the object), the acoustic matching layer 1122 is positioned intermediately between the piezoelectric element 1126 and the subject. The acoustic matching layer 1122 typically includes a combination of different resin materials which modulate the acoustic impedance of the ultrasonic waves generated at the piezoelectric element 1126 to more closely match that of the object being imaged. The lens 1114 helps to concentrate and focus the ultrasonic waves entering and exiting the ultrasound probe, thereby enabling clear cross-sectional and regional imaging of the object.

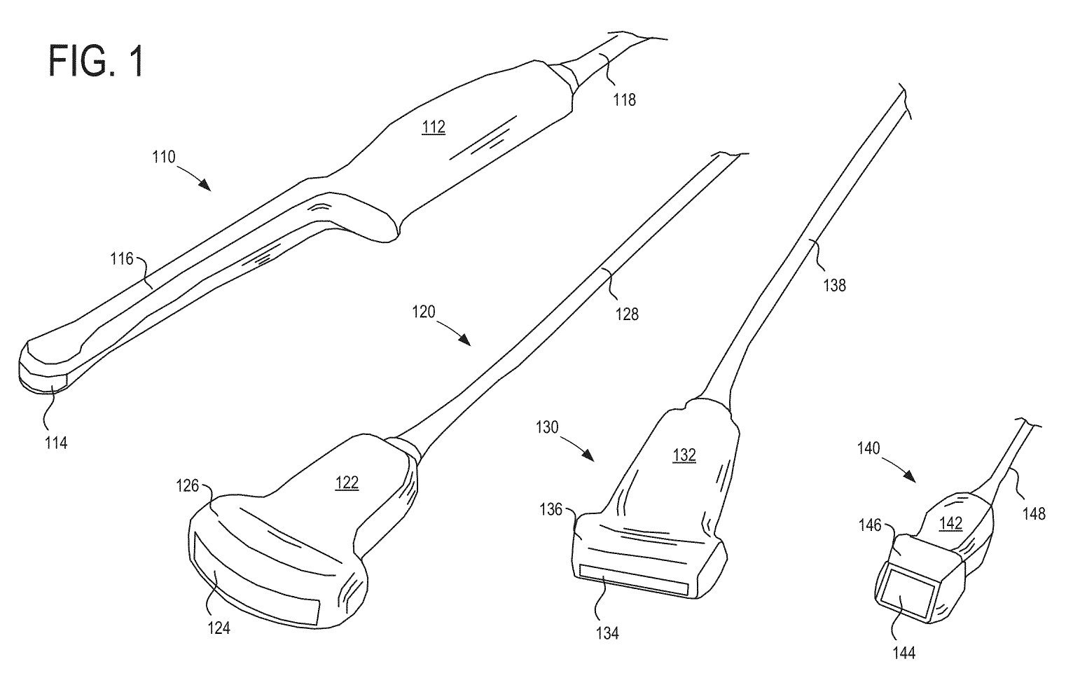

Turning now to FIG. 1, it illustrates several examples of conventional ultrasound probes 110, 120, 130, and 140. Ultrasound probes 110, 120, 130, and 140 respectively comprise handles 112, 122, 132, and 142, lenses 114, 124, 134, and 144, tips 116, 126, 136, and 146, and cables 118, 128, 138, and 148. As illustrated, ultrasound probes can differ in size and shape, lens geometry, and handle style, depending on the clinical application. For example, the long, slender tip 116 of ultrasound probe 110 facilitates insertion into body openings such as for imaging the esophagus. The curved lens 124 may aid in increasing a field of view for imaging larger organs or regions in the body. Furthermore, the curved shape allows for packing a higher density of piezoelectric elements into the probe, which can improve image quality and penetration. The squareish shape of lens 144 is a phased array probe and can be used in sweeping across image regions for generating real-time displays over the image region, such as for imaging blood flow. The smaller footprint of the lens 144 allows the probe to image in constrained locations such as between rib bones. The linear lens 134 allows for a thinner head profile and emits high frequency (shallow penetration depth) ultrasonic waves; it is thus commonly used for shallow depth, high quality imaging. The handles 112, 122, 132, and 142 are generically shaped, with rounded edges, and roughly cylindrical bodies, including a tapering region near the probe tip, which can aid aiming, angling and pressing the probe into the patient's body. Cabling 118, 128, 138, and 148 extends outward from the probe at the opposite end from the lens, which decreases interference between the cable and the imaging. The cabling is coupled to a computer processor and enables transmission and reception of data and signals between the probe and a computer for generating and interpreting the ultrasound images.

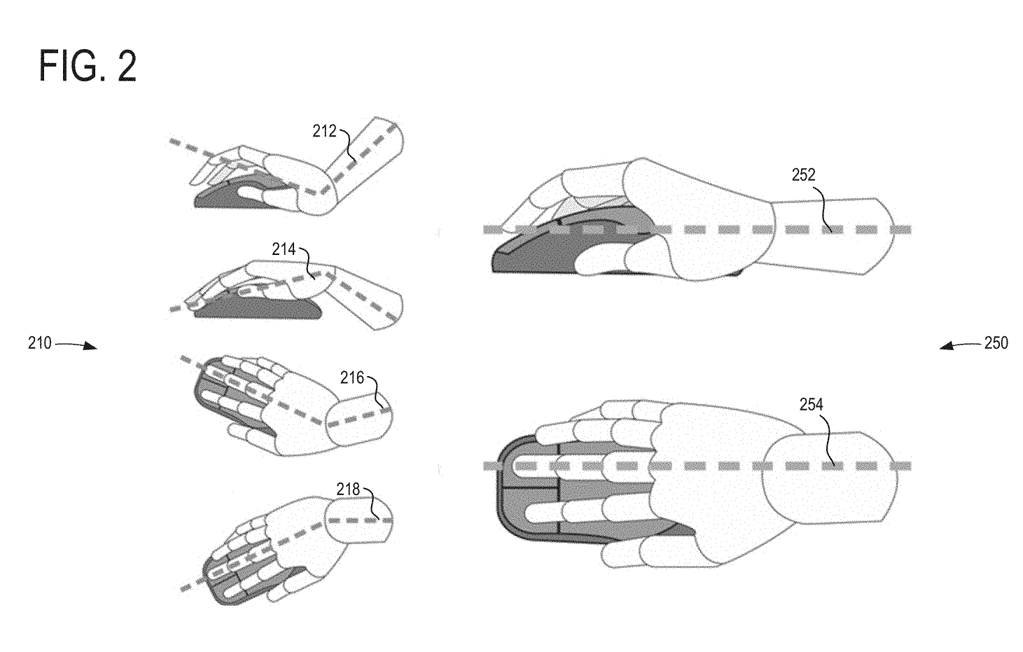

Turning now to FIG. 2, it illustrates various example neutral and non-neutral hand and wrist postures 250 and 210, respectively, in the context of operating a mouse. Neutral wrist posture 250 illustrates linear positioning of the wrist as shown by the linear dashed lines 252 and 254 extending from the forearm through the wrist to the hand. Non-neutral wrist postures 210 illustrate angular (non-linear) positioning of the wrist as shown by the non-linear dashed lines 212, 214, 216, and 218. Non-neutral wrist posture can arise from dorsal (212) and ventral (214) wrist deflection, lateral (216) and medial (218) wrist deflection, or a combination thereof. Other types of non-neutral wrist postures include supination and pronation, where the hand is in a twisted position relative to the forearm. In the context of ultrasound imaging, adopting non-neutral wrist postures when grasping an ultrasound probe can increase the risk of fatigue and chronic strain-related injuries. In contrast, neutrally positioning the wrist while grasping an ultrasound probe can reduce the risk of fatigue and strain-related injuries. Furthermore, similar to ergonomic devices such as a split keyboard for typing, partially or fully custom-fit handles for ultrasound probes can encourage neutral positioning of the wrists and hand while grasping the ultrasound probe, and can relieve stress and pressure on the hand and wrist, thus increasing comfort while reducing risk of ergonomic strain while utilizing the ultrasound probe.

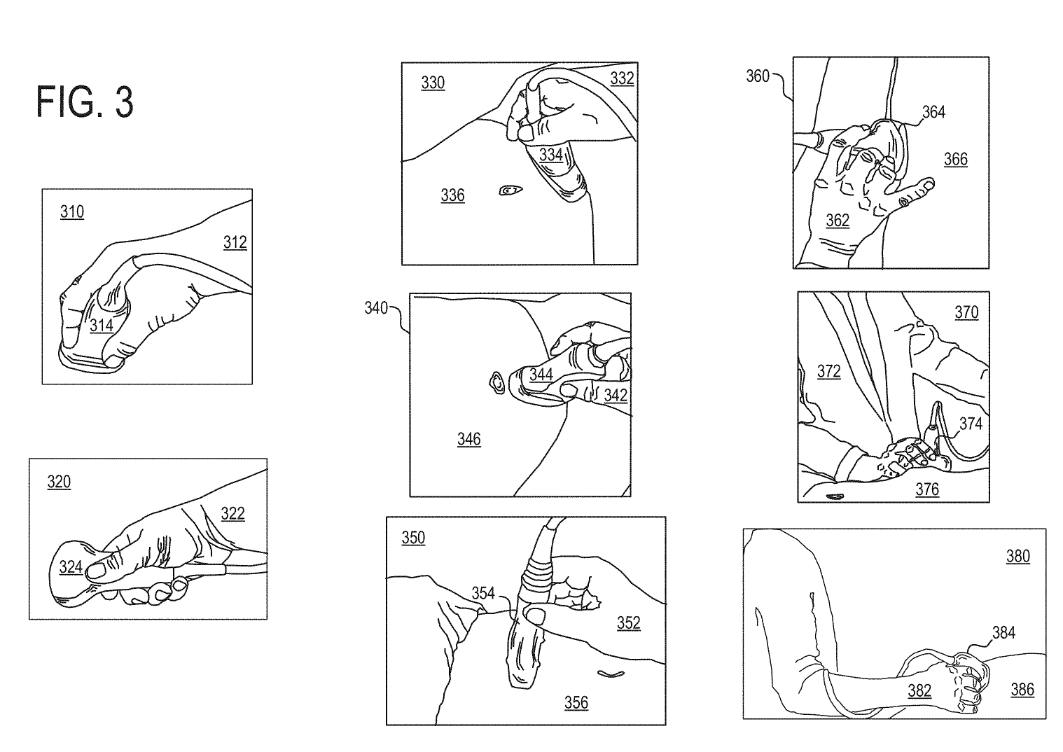

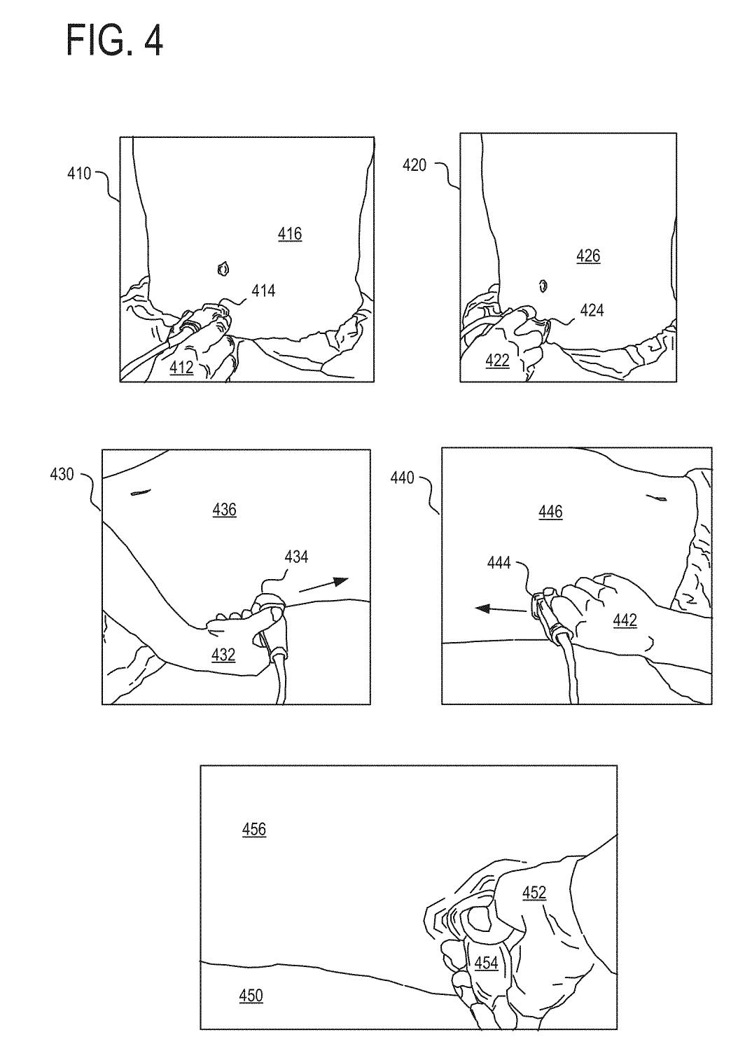

Turning now to FIGS. 3 and 4, they illustrate various hand and wrist postures commonly used by ultrasound imaging operators while grasping a conventional (having non custom-fit handles) linear lens and a conventional square-lens type of ultrasound probe, respectively, during ultrasound imaging. The hand and wrist posture adopted by the operator for the ultrasound imaging can depend on the operator, the type of probe, and the application (e.g., type of ultrasound image or object to be scanned), among other factors. For example, FIG. 3 illustrates eight different variations of hand postures 310, 320, 330, 340, 350, 360, 370, and 380, while grasping an ultrasound probe with a linear lens. The ultrasound probe may be grasped by an operator 312, 322, 332, 342, and 352 using hand postures 310, 320, 330, and 350, respectively, where the thumb and index finger are primarily used to steer or angle the probe tip. In contrast, operators 362, 372, and 382 utilize hand postures 360, 370, and 380, respectively, involving two or more fingers in addition to the thumb to grasp the ultrasound probe. Furthermore, the grip may apply pressure to the probe from a top surface of the handle as in posture 330, from the top of probe tip as in postures 310, 360, 370, and 380, or by grasping the sides of the probe as in postures 340 and 350. In some postures 310, 320, 360, and 380, the operator's fingers straddle nearly the entire length of the probe handle, while in other postures 340 and 350, the operator's fingers transversely straddle the probe handle approximately parallel to the scanned surfaces 346 and 356, respectively.

Because conventional ultrasound probe handles are neither sized nor shaped to conform to a particular operator's hand, the force with which the probe is grasped may be higher relative to a probe handle that is custom fit to an operator's hand. Customizing or partially customizing the fit of an ultrasound probe to an operator's hand may further include tailoring tailor making the ultrasound probe, custom-building the ultrasound probe, and designing the ultrasound probe such that it is made-to-measure according to a 3D model and such that a manually grasped surface of the ultrasound probe snugly conforms to the operator's grasping hand. Furthermore, operators may utilize hand and wrist postures involving non-neutral hand, wrist, and finger positioning in order to firmly grasp and stabilize the ultrasound probe. For example, in posture 360, the fingers of operator 362 are asymmetrically positioned; in particular, the pinky is spread non-neutrally away from the hand in order to stabilize the probe position, introducing strain into the hand of the operator. Furthermore, in postures 340 and 350, the grasping force for stabilizing the probe is generated from the thumb and the index finger, rather than the entire hand, which can lead to earlier onset of hand fatigue and strain. Further still, the hand posture 370 shows the wrist dorsally deflected in a non-neutral fatigue-inducing position.

FIG. 4 illustrates five different hand postures 410, 420, 430, 440, and 450 adopted by operators 412, 422, 432, 442, and 452, respectively, for grasping a phased array type ultrasound probe 414, 424, 434, 444, and 454, respectively, with a square shaped lens while scanning a patient 416, 426, 436, 446, and 456, respectively. Because this type of ultrasound probe has a smaller sized handle, the ultrasound probe can be grasped with a few fingers of the hand, and precludes the operators from utilizing the larger hand (e.g. palm) and arm muscles when supporting and stabilizing the probe, thereby increasing onset of hand and finger strain and fatigue. Furthermore, some of the postures 430 and 450 include non-neutral ventral and dorsal deflection, respectively, of the wrist relative to the hand, which can also cause repetitive strain injuries such as carpal tunnel syndrome. In posture 450, the thumb itself is positioned non-neutrally to apply pressure to the probe 452 for contacting the patient 456.

As further discussed below, ultrasound probes incorporating custom-fit or partially custom-fit handles can reduce operator hand strain and fatigue by sizing and shaping the handle of the probe to fit and conform to an operator's hand, including the palm and fingers, when the operator grasps the ultrasound probe for manipulating, stabilizing, and positioning the probe. For example, the size of the probe handle may be increased in length to match the width of an operator's palm, while maintaining the dimensions and geometry of the lens and tip so that operator comfort and ergonomics can be improved while maintaining imaging resolution, penetration, field of view, ability of the probe to reach and image constrained positions such as between rib bones, and other capabilities. Furthermore, the probe handle can incorporate customized features such as wells or slots positioned to receive an operator's fingers, which may allow the operator to grip and support the probe more easily, while reducing the applied grip force. Further still, providing a tapered handle diameter may reduce finger strain by matching the tapered handle diameter with the tapered grip diameter from the longer to the shorter fingers of the hand. Further still, utilizing a textured (e.g. ribbed, veined, and the like) grasped surface and/or a tacky rubber, polymer, coating, or adhesive on the grasped surface of the probe handle to increase friction between the handle and the operator's hand may reduce finger and hand strain.

The customization features of the handle such as size, shape, size and number of finger wells, handle tapering, and the like, may be incorporated into the design of the ultrasound probe in varying degrees. Partial customization may include manufacturing ultrasound probes with a predetermined number of classifications. For example, analogous to apparel sizing (e.g., x-small, small, medium, large, x-large, xx-large), partially custom-fit probe handles may include a predetermined number of probe handles sizes may be fabricated to at least approximately fit the most common human hand sizes. As a further example, partially custom-fit probe handles may be fabricated having one of a predetermined number of finger well, a predetermined number of finger well sizes, and a predetermined set of different finger well spacings to at least approximately fit the most common human finger sizes and spacing. As a further example, partially custom-fit probe handles may be fabricated to have one of a predetermined number of handle tapering extents to at least approximately fit the most common human hand finger grip tapering extents. Handles may be further partially-customized by specifying the probe type from a predetermined number of probe types (e.g., types of ultrasound probes 110, 120, 130, 140, and the like), the ultrasound imaging application (from a predetermined number of common clinical applications such as maternal abdomen, kidney, vaginal, esophageal, or cardiac ultrasound imaging, and the like), which may constrain the basic handle shape to be selected from a set of predetermined handle shapes for a particular application.

Handles may be further partially-customized by the grasping position (from a predetermined number of positions commonly employed by operators), which may constrain the basic handle shape, finger well orientation and number, and other features based on a set of predetermined handles for a particular grasping position. For example, five types of grasping positions 410, 420, 430, 440, and 450 are showing in FIG. 4 to be associated with a phased array type ultrasound probe 140. As a further example, eight different grasping positions 310, 320, 330, 340, 350, 360, 370, and 380 are shown to be associated with a linear lens ultrasound probe 134. Other grasping positions for the ultrasound probes 140 and 134 and other grasping positions for other types of ultrasound probes may be included in the predetermined grasping positions. In this way, fabricating partially customized probe handles may provide some degree of customization (thereby reducing ergonomic strain) while reducing the manufacturing costs relative to manufacturing custom-fit probe handles. As shown in FIGS. 3 and 4, each grasping position may be classified by the number of fingers grasping the ultrasound probe, the particular fingers grasping the ultrasound probe, the orientation of the ultrasound probe relative to the grip (e.g., transverse orientation for postures 330 and 350; longitudinal orientation for postures 340, 360, 370; axial orientation for postures 310, 320, 410, 440, and 450), and by other classification types.

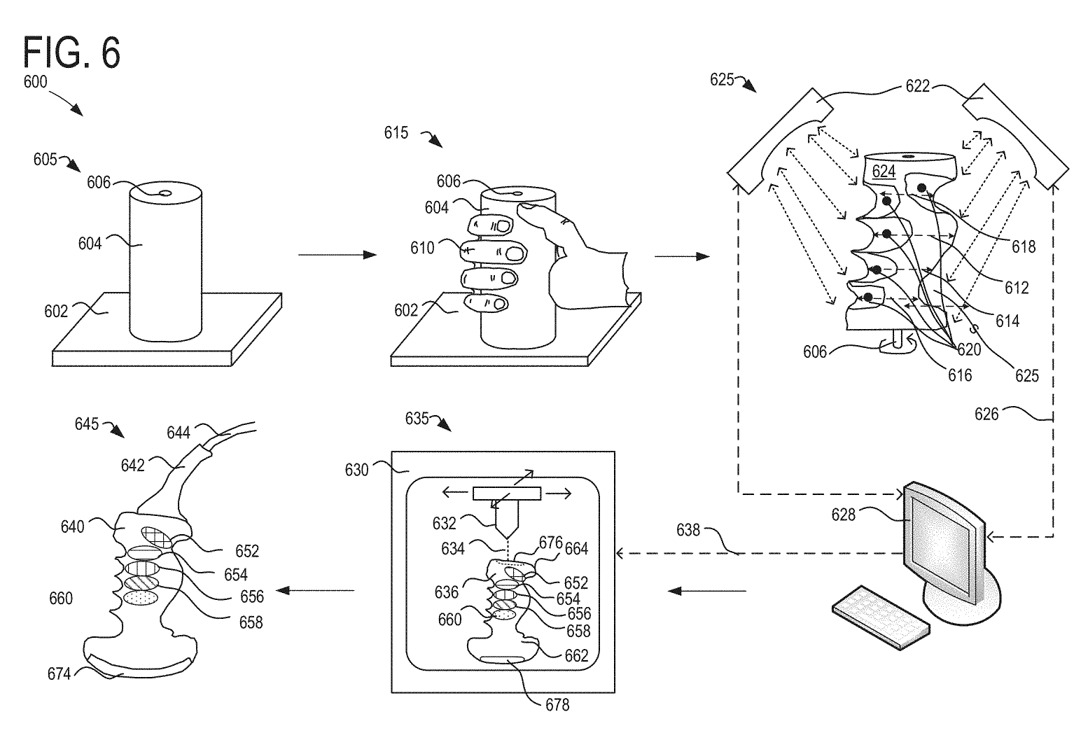

Turning now to FIG. 6, it illustrates a process flow schematic 600 for manufacturing a customized handle for an ultrasound probe. At step 605, a volume of impressionable material 604 removably mounted on a rigid base 602. The impressionable material 604 may include a rod positioned through its longitudinal axis. The impressionable material 604 may include materials that may be non-elastically deformed when grasped with the hand such that an impression of the grasping hand is imprinted into the impressionable material 604 when the impressionable material 604 is released from the grasped hand. The impression of the grasping hand may include fingerprints, palm prints, and divots, depressions, finger wells and palm wells impressed into the impressionable material 604. As examples, the impressionable material 604 may include clays, foams, viscoelastic polymers, gels, plasticene, various modeling compounds, and other viscoelastic materials that can be deformed non-elastically and that stably hold their deformed shape and state after being manually grasped. As a particular example, the impressionable material may include a gel within a flexible membrane; upon squeezing the gel and/or warming the gel by grasping the impressionable material, a chemical reaction may be initiated in the gel that causes the gel to solidify over a duration during which the impressionable material is grasped, thereby forming a solid physical impression conforming to an operator's grasping hand. The size and dimensions of the impressionable material 604 should be large enough to be grasped by a range of common human hand sizes. Step 615 depicts a human hand 610 grasping the impressionable material 604 mounted on the base 602. The posture of the grasping hand 610 grasping the impressionable material 604 may correspond to a grasping position or posture used by an ultrasound probe operator for holding an ultrasound probe while conducting an ultrasound exam. For example, the grasping posture of the hand 610 may correspond to postures 310, 320, 330, 340, 350, 360, 370, 380, 410, 420, 430, 440, 450, or other postures employed by ultrasound probe operators for grasping ultrasound probes.

At step 625, the impressionable material 604 having been deformed and imprinted with the grasping hand from step 615 is shown as a physically imprinted model 624 of the grasping hand posture. The physically imprinted model 624 comprises a three-dimensional replica of the grasping hand posture; the outer surface of the physically imprinted model 624 is the negative surface conforming to the positive surface of the grasping hand 610, including finger and thumb wells 620 formed by the depressed fingers and thumb of grasping hand 610, and palm well 625 formed from depressing the palm of grasping hand 610 into the impressionable material 604. The black dots shown in the finger and thumb wells 620 may correspond to the finger and thumb pad positions of grasping hand 610 and may represent the three-dimensional positions of specific contact pressure points of the grasping posture of grasping hand 610 on the outer surface of the physically imprinted model 624. Incorporating the three-dimensional positions of the finger and thumb pads in the hand grasping posture for grasping an ultrasound probe may aid providing proper positioning of the finger and thumb pads when grasping a custom-fit or partially custom-fit ultrasound probe, which can reduce operator strain while increasing grasping force by increasing the friction between an operator's finger and thumb pads and the grasped surface of the ultrasound probe. Similarly, incorporating the three-dimensional position of the palm in the hand grasping posture by incorporating the palm well 625 into the grasped surface of the ultrasound probe may aid providing proper positioning of the palm when grasping a custom-fit or partially custom-fit ultrasound probe, which can reduce operator strain while increasing grasping force by increasing the friction between an operator's palm and the grasped surface of the ultrasound probe.

In addition to the finger and thumb wells 620, the physically imprinted model 624 may include a tapered grasped diameter, as represented by the dashed double arrows 612, 614, and 616. In the example illustrated in step 625, the length of dashed double arrow 616 is less than the length of dashed double arrow 614, which is less than the length of dashed double arrow 612. Dashed double arrow 616 corresponds to the axial position of the pinky finger well 620 (as is evident from comparing steps 615 and 625) and is less than dashed double arrow 614, which corresponds to the axial position of the ring finger well 620 (as is evident from comparing steps 615 and 625) since the length (and grasping diameter) of the pinky finger is less than the length of the ring finger. Similarly, dashed double arrow 614 is less than dashed double arrow 612, which corresponds to the axial position of the middle finger well 620 (as is evident from comparing steps 615 and 625) since the length of the ring finger is less than the length of the middle finger. Accordingly, the diameters 612, 614, and 616 may be accordingly sized (thereby tapering the physically imprinted model 624) to correspond to the lengths of the fingers of the operator's hands 610. Furthermore, the topmost finger wells 620 positioned on either side of the physically imprinted model 624 correspond to the grasping positions of the operator's thumb and forefinger (index finger). The grasping diameter 618 of the physically imprinted model 624 at the grasping positions of the thumb and forefinger may be larger relative to the diameters 612, 614, and 616 because the combined length of the thumb and forefinger is longer than the individual middle finger, ring finger, and pinky fingers. Conversely, the grasping diameter of the physically imprinted model 624 may be tapered from diameter 612 to diameter 618 because the grasping force of the ultrasound probe by the thumb and the forefinger is higher than the grasping force of other fingers. Accordingly, the diameter of the physically imprinted model 624 at any particular position may be representative of the finger length and/or the grasping force of one or more fingers when holding an ultrasound probe.

At step 625, the physically imprinted model 624 is three-dimensionally (3D) scanned using one or more scanning devices 622 positioned peripherally (above, and/or below and/or adjacent) to the physically imprinted model 624 to obtain enough data regarding the shape and appearance in order to construct a digital 3D model of the operator's manual (hand) posture while grasping the ultrasound probe. The 3D scanning devices 622 may be hand-held scanning devices or non hand-held scanning devices. The physically imprinted model 624 may additionally be rotated about its axis, for example by mounting and rotating rod 606 on a rotating base, which can facilitate and expedite the 3D scanning process. 3D scanning may employ various 3D scanning technologies such as time-of-flight 3D laser scanning, triangulation 3D laser scanning, conoscopic holography, structured-light 3D scanning, modulated light 3D scanning, as well as non-contact passive 3D scanning technologies using photography such as stereoscopic photography, photometric systems, and silhouette techniques. Furthermore, user assisted image-based modeling methods may employ commercial software packages such as D-Sculptor, iModeller, Autodesk ImageModeler, 123DCatch, PhotoModeler, and the like, combined with a provided set of measured manual attributes to build a 3D model of the ultrasound probe operator's grasping hand. For example, multiple photos of the ultrasound probe operator's grasping hand in a grasping position may be obtained from several different points in 3D in order to build a 3D digital replica of the operator's grasping hand. Further still, techniques such as computed tomography, microtomography, and magnetic resonance imaging may be used to construct a 3D digital replica of the operator's grasping hand by obtaining and stacking or volume rendering a series of 2D cross-sections of the operator's grasping hand. At step 625, the 3D scanning data is transmitted via signals 626 to a computer processor 628 where a 3D digital model is rendered or digitized from the 3D scanning data. Rendering or digitizing a 3D model may include generating a point cloud 3D digital model of the operator's grasping hand utilizing one or a combination of 3D scanning technologies.

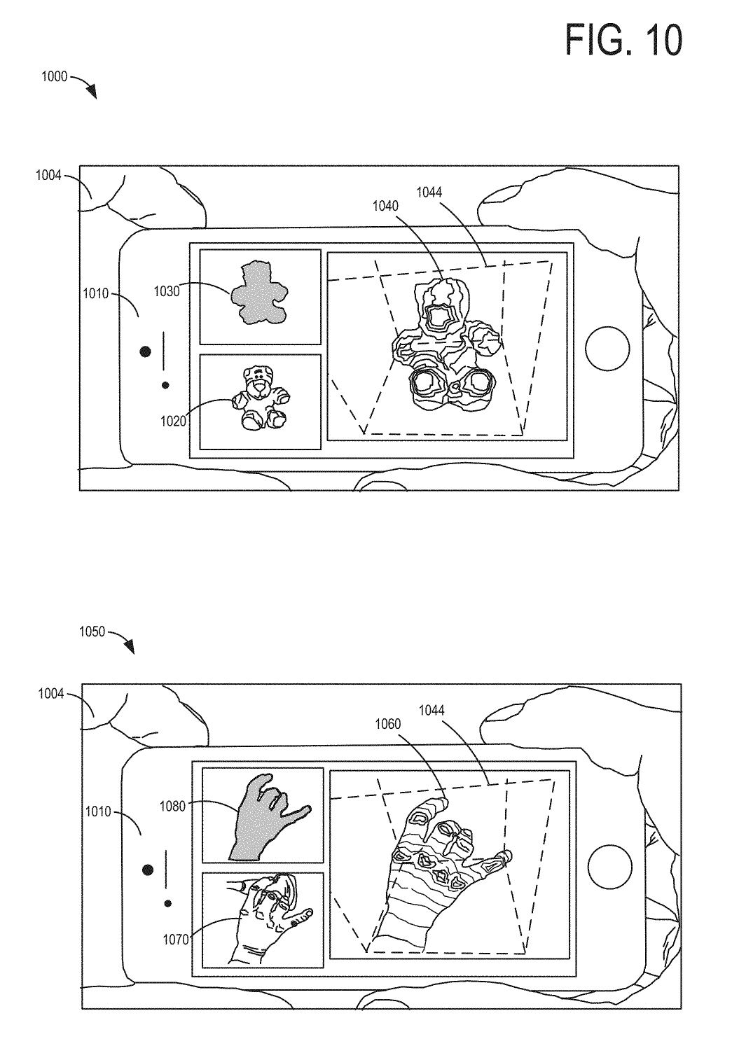

Turning now to FIG. 10, it illustrates a schematic 1000 for an example of a 3D scanning technology, utilizing a 3D scanning application (app) such as Microsoft MobileFusion on a computer such as a mobile device 1010 including a digital camera. Generating the 3D model of an object 1020, may involve capturing a series of photographs and/or videos of an object 1020 by panning around the object 1020 in a 3D volume space 1044 and then producing a 3D digital rendering 1040 (digitizing) of the object 1020. The app may densely track the position of the mobile device 1010 relative to the object 1020 in 3D during the panning around the object 1020 by comparing live red green blue (RGB) light data 1030 received by the mobile device camera. Each live frame may be stereomatched relative to previous and later frames to compute a 3D depth map rendering 1040 of the object 1020. The stereo depth maps may then be rendered or meshed into a 3D model of the object 1020. Additional 3D photo and/or video scans of the object may be captured to augment and refine the 3D model.

Schematic 1050 illustrates a user 1004 using such a mobile device app to generate and digitize a 3D model of an ultrasound probe operator's grasping hand 1070 in a posture 362 used to grasp a particular type of ultrasound probe 364. For example, the user 1004 may pan the mobile device 1010 around the grasping hand 1070 to capture a series of photos and/or video of the grasping hand 1070 within a 3D volume space 1044. The app may then compute a 3D depth map 1060 rendering of the grasping hand 1070 by stereomatching a series of RGB light data 1080 received by the mobile device camera. These stereo 3D depth maps 1060 may then be digitized into a 3D digital model of the grasping hand 1070. As another example, the user 1004 may use the mobile device app to directly generate and digitize a 3D model of a physically imprinted model 624 of a manually grasped surface.

In some examples, the 3D digital model can be used to determine a set of manual attributes of the ultrasound operator's grasping hand such as finger and thumb lengths, palm widths hand span, grasping hand diameter, and the like. Alternately, the 3D digitized models can be augmented with manual attribute data determined by physically measuring the operator's hand. In still further examples, a 3D digitized model of an ultrasound probe user's grasping hand may be constructed from manual attribute data determined from physically measuring the operator's hand and inputting the measured data (as model parameters) into a parameterized template model of a user's grasping hand. Upon specifying the parameterized template model with measured data model parameters, a partially customized 3D model of the operator's grasping hand may be rendered.

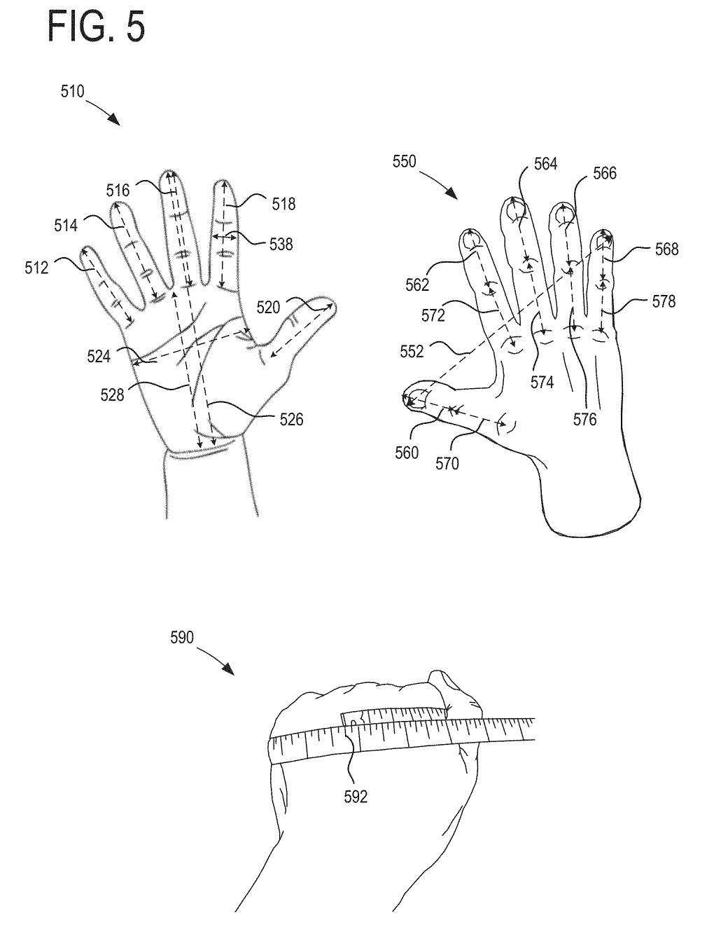

Turning now to FIG. 5, it illustrates various examples of manual attributes using schematics of a palm-up open hand 510, a palm-down open hand 550, and a fisted hand 590. The manual attributes can be used to augment 3D modeling and rendering of an ultrasound probe user's grasping hand, as discussed above with reference to FIGS. 6, 7, and 10. The manual attributes can include finger and thumb lengths 512, 514, 516, 518, and 520, which can aid in modeling grasping diameters and grasping forces for each finger and thumb, the dimensions of each finger and thumb wells, and the like. The manual attributes can further include finger widths or diameters 538 and a hand length 526. The finger diameters 538 may aid in accurately modeling finger well widths and grasping forces for each finger, while the hand length 526 may aid in more accurate modeling of the diameter of the manually grasped surface. The manual attributes can further include finger segment lengths 560, 562, 564, 566, and 568, between the finger and thumb tips and the first knuckles; finger segment lengths 570, 572, 574, 576, and 578, between the first knuckles and the second knuckles; and or the relative ratios thereof. Finger segment lengths can aid in modeling the depths and dimensions of the finger wells along each length of the finger/thumb. The manual attributes can further include a hand circumference 592, measured as illustrated for the fisted hand 590. The hand circumference 592 may aid in modeling the diameter as well as the overall dimensions of the manually grasped surface. Other manual attributes not depicted in FIG. 5 may also be determined and used for augmenting the 3D modeling of the operator's hand such as finger tip shape and with, and the like.

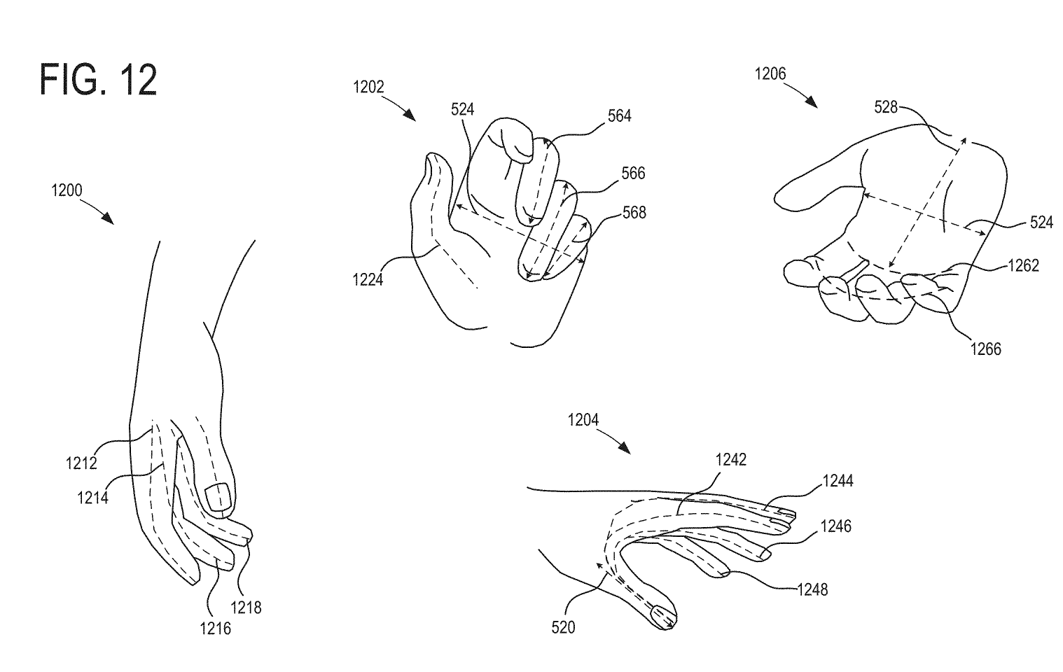

Turning now to FIG. 12, it illustrates a schematic of a human hand in various relaxed at-rest postures. In some examples, manual attributes for augmenting 3D models of the operator's grasping hand may be determined by imaging and/or digitizing hands in relaxed at-rest postures. Some example relaxed at-rest postures include where the hand is pointing downwards 1200, pointing upwards 1202, extended horizontally palm-up 1206, and extended horizontally palm-down 1204. As shown in posture 1200 and 1202, the fingers of the hand may slightly curl when the hand is relaxed, the fingers curling more so when the hand is pointing upwards as in posture 1202 due to the added influence of gravity. In both postures 1200 and 1202, the forefinger remains straighter than the other fingers, with the little pinky finger being curled more so than the other fingers. Manual attributes such as the palm width 524, palm length 528, and various finger segment and finger lengths including finger segment lengths 564, 566, 568, and finger length 520 may be obtained from the relaxed at-rest postures. Furthermore, other manual attributes such as finger length arcs 1212, 1214, 1216, 1218, and 1224 may be measured. In one example, finger segment lengths and finger lengths may be estimated from their respective finger length arcs. Furthermore, finger length arcs may be used to determine finger grasping lengths in the 3D model of the operator's grasping hand. Turning to relaxed at-rest posture 1206, palm width arc 1262 and finger span arc 1266 are manual attributes that may further aid in augmenting 3D operator grasping hand models, such as accurately mimicking the manually grasped surface topography and dimensions. Similarly, the finger-thumb arc lengths 1242, 1244, 1246 and 1248 are manual attributes that may further aid in augmenting 3D operator grasping hand models, such as accurately mimicking the manually grasped surface topography and dimensions. In this way, manual attributes from both relaxed at-rest hand postures (as shown by example postures in FIG. 12) and non-relaxed postures (e.g., such as those illustrated in FIG. 5) may be used to determine various manual attributes for augmenting the 3D operator's grasping hand models.

Returning to FIG. 6, after generating and digitizing the 3D model of the operator's grasping hand at step 625, the computer processor 628 may generate a 3D model of a manually grasped surface 636 corresponding to the digitized 3D model of the operator's grasping hand. The manually grasped surface 636 may include a negative surface that conforms to the positive surface of the operator's grasping hand, as specified by the 3D digitized model. In other words, when the manually grasped surface 636 may be brought together and mated with the operator's grasping hand, the free volume (empty space) between the manually grasped surface and the operator's grasping hand is largely reduced and negligible relative to the free volume between an operator's grasping hand and a conventional non-custom-fitted (or non-partially custom-fitted) ultrasound probe. The bounds of the 3D model of the manually grasped surface 636 can extend beyond the bounds of the operator's grasping hand (or 3D model thereof) in order to facilitate grasping of a fabrication or physical facsimile of the manually grasped surface 636. For example, as shown in FIG. 6, the manually grasped surface has lipped portions 664 and 662 above and below the topmost finger wells that diametrically extend beyond the diameter of the grasping hand fingers. In this way, the lipped portions 662 and 664 may aid in securing an operator's grasp on the custom-fitted ultrasound probe by reducing slippage of the operators hand in an axial direction (upwards or downwards), and thereby reducing operator strain while maintaining the operator's grasping force applied to the ultrasound probe. Accordingly, the manually grasped surface 636 corresponds to the external surface of a custom-fitted ultrasound probe that is grasped by an operator's hand while performing an ultrasound scan.

The computer processor 628 may transmit the 3D model data 638, including the 3D model data of the operator's grasping hand and/or the 3D model data of the manually grasped surface) to a 3D printing device 630. At step 635, the 3D printing device may translate a printer head 632 three-dimensionally, utilizing to the 3D model data 638, while dispensing curable printing media 634 in order to create a 3D replica of the manually grasped surface 636. As shown in FIG. 6, the printed 3D replica of the manually grasped surface 636 can include customization features such as finger wells 620, tapering of the handle diameter, and textured surfaces on at least a portion of the manually grasped surface 636. For example, at least a portion of the finger wells 620 may be ribbed (having raised ribs or other structures) in order to increase friction between an operator's hand and an ultrasound probe handle when an operator grasps the manually grasped surface 636. The ribs may be oriented approximately parallel to the longitudinal direction of the grasped fingers (as shown by ribs 654), perpendicular to the longitudinal direction of the grasped fingers (as shown by ribs 654), oblique to the longitudinal direction of the grasped fingers (as shown by ribs 656), or a combination of one or more thereof (as shown by ribs 664). The textured surface can also include raised dots as shown by the textured finger well region 660. Other textured surfaces, including combinations thereof, incorporating raised or depressed surface structures can be utilized to increase friction between an operator's grasping hand an the grasped surface of a custom-fitted ultrasound probe. The replica of the 3D manually grasped surface 636 may also be hollow, as indicated by the openings 676 and 662 that may be connected by a continuous cavity or channel.

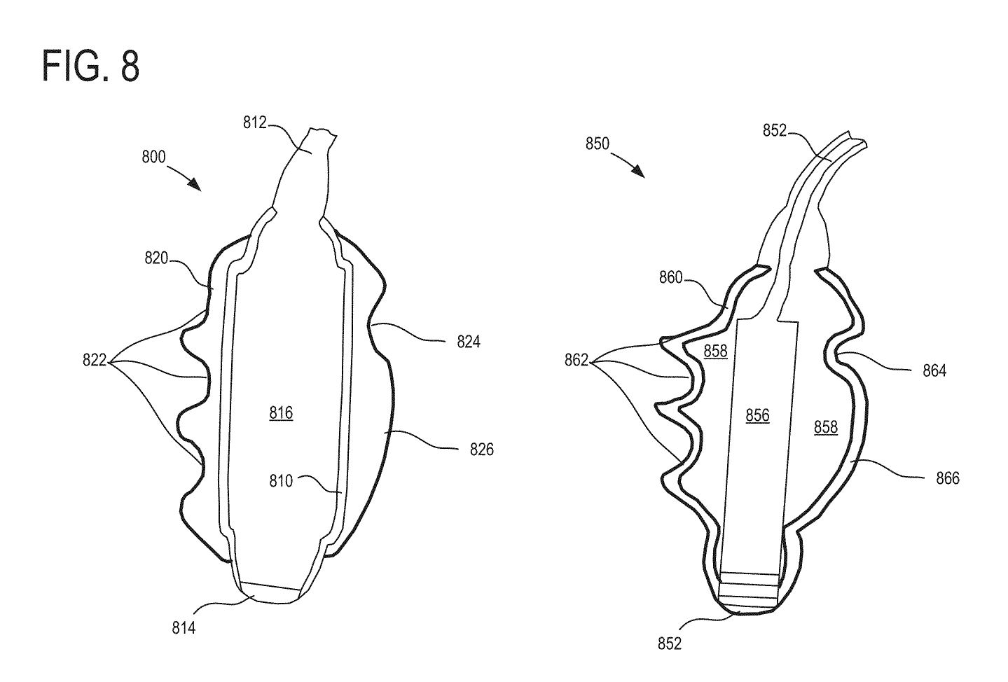

At step 645, the custom-fitted ultrasound probe 640 may be assembled by coupling the facsimile of the manually grasped surface 636 to the probe lens 674, cabling 642, and probe electronics such as the probe transducer components (as discussed above with reference to FIG. 11). Furthermore, assembling the ultrasound probe 640 may include inserting the probe electronics and transducer components within the cavity of the hollow manually grasped surface 636. Assembling the ultrasound probe 640 may also include removably attaching the manually grasped surface 636 to the remaining components of the ultrasound probe 640. For example, the probe electronics and transducer components may be removable inserted into the cavity of the hollow manually grasped surface 636. Furthermore, the probe lens 674 may be removably coupled to the manually grasped surface 636. Removably attaching the manually grasped surface 636 to assemble the ultrasound probe 640 may reduce ultrasound probe repair and replacement costs since the manually grasped surface 636 can be removed and replaced, or removed to access and replace other probe components. As discussed further with reference to FIG. 8, the manually grasped surface 636 may include a hollow flexible sleeve or a hollow rigid structure. Accordingly, the curable printing media 634 may be selected to provide a manually grasped surface 636 that is flexible or rigid, smooth or rough, tacky or untacky, and other various desired characteristics.

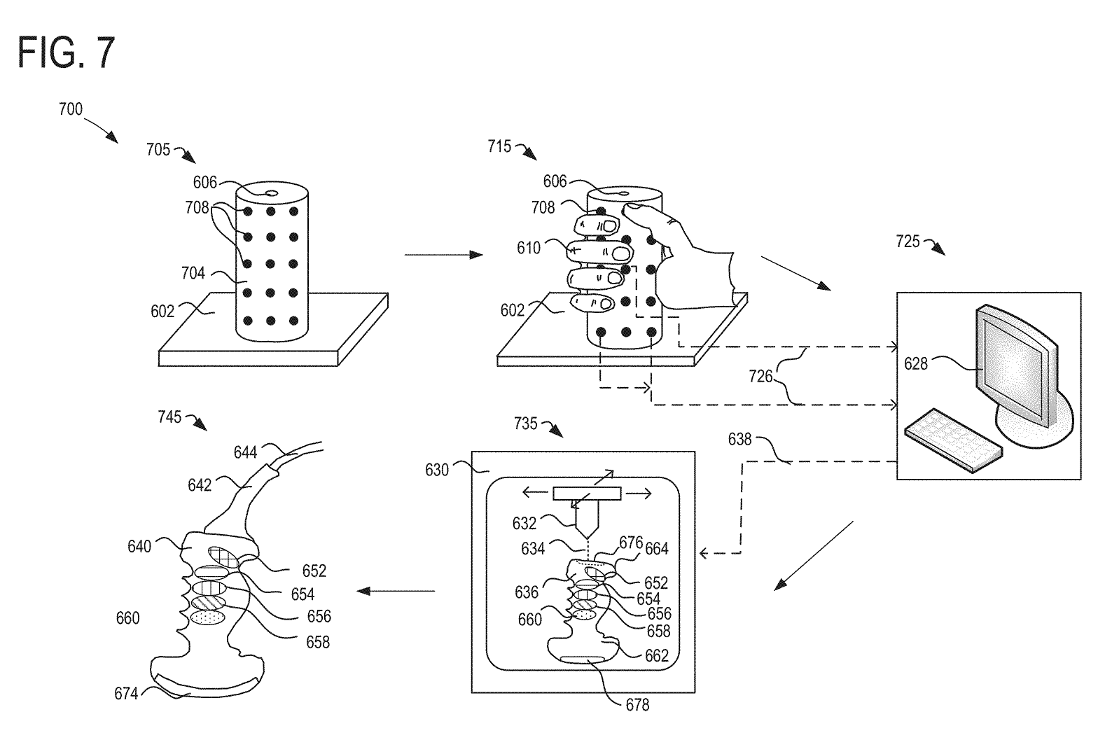

Turning now to FIG. 7, it illustrates a schematic 700 of another example process flow for 3D modeling and digitally rendering the 3D model of an ultrasound probe user's grasping hand 610. In contrast to the impressionable material 604 utilized in FIG. 6 to generate a physical imprinted model 624 of the grasping hand, the process flow of FIG. 7 illustrates an ultrasound probe template 704 including a plurality of contact sensors 708 positioned at an exterior surface thereon. The probe template 704 may comprise a basic geometric shape such as the cylindrical form depicted in FIG. 7, however in other embodiments, the probe template 704 may comprise a non-symmetrical irregular 3D shape. For example, the probe template 704 may incorporate one or more customization features such as finger wells, palm wells, tapering of the grip diameter, and the like. In another example, the probe template may include an impressionable material surrounded by a flexible membrane having a plurality of contact sensors distributed thereon. In this way, the probe template may conform to an operator's grasping hand (upon being grasped), while determining grasping pressures and contact points for input into the 3D model.

Various probe templates 704 may be fabricated, each probe template 704 suitable for generating a 3D model of an ultrasound probe operator's grasping hand for a particular type of ultrasound probe. The probe templates 704 may be fashioned taking into account the type of ultrasound probe (as discussed above with reference to FIG. 1), and the typical grips used by operators for holding those ultrasound probes (as discussed above with reference to FIGS. 3 and 4). An advantage of a probe template 704 having a basic geometric shape is that the probe template 704 may generic enough to model an operator's hand grasping different types of ultrasound probes and the probe template 704 may be inexpensively manufactured relative to a probe template 704 having a non-symmetrical irregular 3D shape. However, 3D models of an operator's grasping hand generated employing probe templates 704 having a non-symmetrical irregular 3D shape may be more accurate and precise as compared to 3D models generated employing probe templates 704 with basic geometries.

In some examples, the contact sensors 708 may be distributed in a regular array across the external grasped surface of the probe template 704. In other examples, the contact sensors 708 may be positioned and concentrated at locations on the external grasped surface of the probe template 704 corresponding to and facilitating the determination of certain manual attributes of the operator's grasping hand. For example, contact sensors 708 may be positioned at regions where an operator's grasping hand's finger and thumb tips may be located in order to better estimate finger lengths and grasping diameters of the operator's hand. In another example, contact sensors 708 may be positioned at regions near the periphery of an operator's grasping hands in order to better estimate the bounds of the operator's hand (e.g., palm width, and the like). Increasing the density of the array of contact sensors 708 may aid in raising the precision and accuracy of the 3D model.

At step 715, the operator grasps the probe template 704 with their hand 610. The contact sensors 708 may be configured to sense both the positions of the points of contact of the probe template 704 with the operator's hand 610 as well as the pressure or force at each contact point. Determining the pressure at each contact point may aid in generating a more accurate 3D model of the manually grasped surface. For example, if the contact sensors 708 detect a higher pressure at contact points related to the grasping forefinger and thumb relative to the contact points corresponding to the grasping middle finger, the forefinger and thumb wells in the resulting manually grasped surface 636 may be made deeper than the middle finger well. The contact sensors 708 may be configured to transmit contact point position and pressure data to the computer processor 628 via signals 726.

At step 725, the computer processor 628 generates and renders a 3D model of the operator's grasping hand from the transmitted contact point position and pressure data via signals 626. Generating and rendering/digitizing a 3D model of the operator's hand and the corresponding manually grasped surface may include generating a point cloud 3D digital model of the operator's grasping hand utilizing one or a combination of 3D scanning/rendering technologies, as discussed above with reference to FIGS. 5, 6, and 11. The computer processor 628 then transmits the 3D model data of the manually grasped surface and/or the operator's hand via signals 638 to a 3D printing device 630. The process flow continues at steps 735 and 745, which may be analogous to steps 635 and 645 of FIG. 6.

Turning now to FIG. 8, it illustrates cross-sectional views of two embodiments 800 and 850 of a custom-fit ultrasound probe. Custom-fit ultrasound probe 800 includes a conventional ultrasound probe 816 surrounded by a hollow and flexible custom-fit sleeve 820, cabling 812, and lens 814. Custom-fit sleeve 820 (or sheath, cover, “koozie”, and the like) may comprise a manually grasped surface including finger and thumb wells 822 and 824 and palm contacting surface 826. The custom-fit sleeve 820 may further include textured and/or coated external surfaces to increase friction between an operator's grasping hand and the custom-fit sleeve 820. Furthermore, the manually grasped surface may include textured and/or coated interior surfaces to provide for increased friction between the interior surfaces and the conventional ultrasound probe 816 inserted therein. The coating may increase a friction coefficient (e.g., tackiness) of the surfaces of the sleeve. For example, when the operator's hand grasps the custom-fit sleeve 820, the custom-fit sleeve may flex and contact and grip the exterior shell 810 of the ultrasound probe 816, thereby reducing operator strain when manipulating the ultrasound probe 816. In other embodiments, the custom-fit sleeve may include a partially customized sleeve and a rigid sleeve. In this way, a conventional ultrasound probe may be easily retrofitted with a custom-fit or partially custom-fit hollow sleeve 820 in order to reduce operator strain while conducting ultrasound exams.

Custom-fit (or partially custom-fit) ultrasound probe 850 includes a rigid hollow probe handle 860 enclosing an ultrasound probe electronic components 856 such as the acoustic matching layer 1122, a piezoelectric element 1126, and a backing material 1120, as well as the probe electronics coupled to the cabling 852. The rigid hollow probe handle 866 has an exterior manually grasped surface that may include finger and thumb wells 862 and a palm-contacting region 866. Furthermore, the probe handle 860 may also enclose interior volumes 858 surrounding the electronic components 856, which may advantageously allow for positioning additional heat dissipation devices such as heat sinks, fins, and the like. Consequently, the custom fit probe handle 860 may allow for increased heat dissipation during ultrasound exams, which can prolong the usable life of the ultrasound probe 850 and may further reduce operator strain. Ultrasound probe 850 may be assembled by inserting (including removably inserting) the electronic components 856 into the probe handle 860, mounting (including removably mounting) the lens 852 at the tip of the probe handle 860 and securing (including removably securing) the cabling 852 at the upper opening of the probe handle 860. Removably mounting, securing and inserting may involve fastening mechanisms such as snapping protrusions into recesses, screwing opposing threads together, friction fitting, quick disconnect connecting, and other mechanisms.

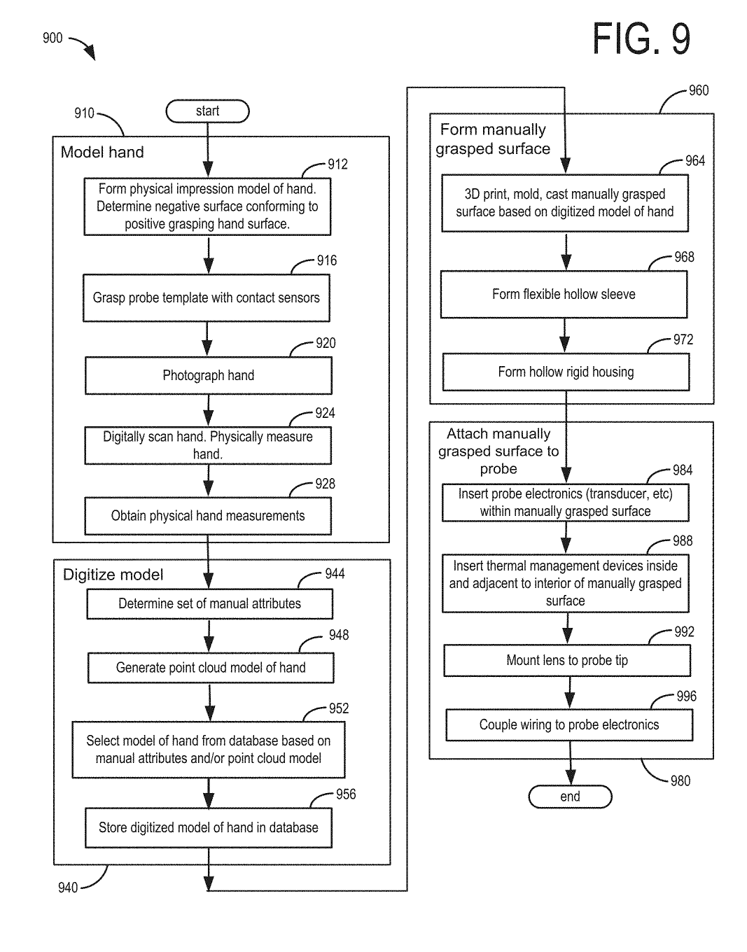

Turning now to FIG. 9, it illustrates a flow chart for a method 900 of manufacturing a custom-fit (or partially custom fit) ultrasound probe. Method 900 begins at 910 by modeling an ultrasound probe operator's hand. Modeling the operator's hand may include 3D modeling the probe operator's hand. At 912, a physical impression model of the operator's hand may be obtained, as discussed above with reference to FIG. 6. Furthermore, a negative surface conforming to the positive grasping hand surface may be rendered from the physical impression model of the operator's hand. Next, at 916, a model of the operator's hand may be generated by grasping a probe template with contact sensors, as described with reference to FIG. 7. Further still, modeling the operator's hand can include taking a series of photographs and/or videos of the hand at 920, as described with reference to FIGS. 6, 7, and 10. Further still, modeling the operator's hand may include digitally scanning the hand and physically measuring one or more manual attributes, as described above with reference to FIGS. 5, 6, 7, and 10. Further still, modeling the operator's hand can include physically measuring one or more manual attributes of the operator's hand at 928, as discussed above with reference to FIGS. 5-6. Modeling the operator's hand may include one or a combination of 912, 916, 920, 924, and 928.

Next, method 900 continues at 940 where the model of the operator's hand is rendered or digitized, including one or a combination of determining the set of manual attributes 944 for specifying the model, and generating a point cloud model of the hand 948, as described above with reference to FIGS. 6, 7 and 10. Furthermore, rendering the model of the operator's hand may include storing the digitized model in a database. Storing digitized 3D ultrasound probe operator hand models in a database may be advantageous because a 3D model of an operator's hand can be utilized when fabricating multiple ultrasound probes for a specific operator. For example, once a 3D hand model has been generated and digitized (and stored in the database), the 3D hand model can be recalled whenever a new or replacement custom fit ultrasound probe or probe handle is desired, such as when a custom fit ultrasound probe of a different type is desired. Storing the 3D hand models in a database thus precludes generating and digitizing a 3D hand model each time a custom fit ultrasound probe is fabricated. The database may also facilitate generating models for an operator that favors a particular grip position when grasping different types of ultrasound probes since the parameter attributes or 3D model for that grip position can be applied when generating 3D grasping hand models for that operator across different types of ultrasound probes.

As another example, the database may be populated with a plurality of predetermined 3D hand models representative of and spanning typical predetermined hand classifications. For example, the plurality of 3D hand models may include models representative of and spanning typical human hand sizes (e.g., x-small, small, medium, large, x-large, and the like), ultrasound probe operator grip positions (e.g., transverse, longitudinal, downward, upward, two-finger, three-finger, and the like), and ultrasound probe types (e.g., linear, phased, curved, interior cavity probes, and the like). As a further example, known or measured manual attributes relating to an operator's hand size (e.g., finger lengths, finger segment lengths, palm width, hand span, hand length, hand circumference, grasping forces, and the like), preferred grip position, probe type, and the like may be input as parameters to specify a parameterized 3D hand model stored in the database in order to generate and render a custom-fit or partially custom-fit 3D hand model to the operator's hand.

Next at 960 a replica or facsimile of the manually grasped surface based on and corresponding to the 3D model of the operator's hand is formed. As described above with reference to FIGS. 6 and 7, and as indicated at 964, the manually grasped surface may be fabricated by 3D printing, casting, molding, and the like, according to specifications of the 3D model of the operator's hand. 3D printing may include fused deposition modeling, poly jet 3D printing, selective laser sintering, binder jetting, and other 3D printing processes. In this way, the manually grasped surface may comprise a negative surface conforming and custom-fitted to the positive surface of the operator's grasping hand. Furthermore, to facilitate fabrication of the manually grasped surface, a computer processor may be used to translate the 3D model of the operator's hand (e.g., positive surface) to a 3D model of the manually grasped surface (e.g., negative surface), which then may be used to 3D print, mold or cast a physical facsimile of the manually grasped surface. As described above with reference to FIGS. 6 and 7, the facsimile of the manually grasped surface may include operator hand-customized features such as finger and thumb wells, palm-contacting regions, a tapering grasping diameter, and textured or coated interior and/or exterior surfaces, as well as other customizing features. In addition, an operator's name or initials may be inscribed or printed on the surface of the probe for identification. Customization of ultrasound probes may further increase useful life since each operator may be encouraged to take ownership and better care for their customized ultrasound probe, thereby reducing operating costs.

At 968, forming the manually grasped surface can include forming a flexible hollow sleeve that can be slipped over a conventional ultrasound probe, thereby retrofitting the conventional ultrasound probe to have a custom-fit or partially custom-fit ultrasound probe handle that reduces operator strain while easing manipulation of the ultrasound probe during ultrasound exams. At 972, forming the manually grasped surface may further include forming a hollow rigid housing for a custom-fit ultrasound probe. As described in FIG. 8, the hollow rigid housing may house probe electronic components such as the acoustic matching layer 1122, a piezoelectric element 1126, and a backing material 1120, as well as the probe electronics coupled to the cabling 852. The hollow rigid housing may also include free volume spaces 858 between the interior surface of the hollow rigid housing and the probe electronic components, which can allow for additional thermal management devices to be included in the ultrasound probe for increasing heat dissipation.

Next at 980, the manually grasped surface may be attached to other probe components to assemble and form the custom-fit ultrasound probe. Attaching the manually grasped surface to the ultrasound probe may include: inserting probe electronic components into a cavity of the manually grasped surface; inserting thermal management devices thermally coupled to the probe electronics into the cavity of the manually grasped surface and directly adjacent to an interior surface of the manually grasped surface; mounting the lens at first opening of the manually grasped surface at a peripheral tip of the manually grasped surface (thereby forming the ultrasound probe tip); and coupling wiring to the probe electronics at a second opening positioned at an opposite end of the probe to the probe tip. Attaching the manually grasped surface to the ultrasound probe may include removably attaching the manually grasped surface to the ultrasound probe. As such, removably attaching the manually grasped surface may include removably inserting the probe electronic components into the cavity of the manually grasped surface, removably inserting thermal management devices into the cavity of the manually grasped surface, removably mounting the lens at the first opening, and removably coupling wiring to the probe electronics at the second opening. Removably refers to a reversible attaching process whereby attaching and detaching the components of the ultrasound probe can be easily performed without damaging the respective components. For example mounting the lens may include screwing a lens into a threaded opening, coupling the probe electronics may include making a quick-disconnect type of connection to the second opening, and the like. As another example, the interior surface and structure of the manually grasped surface may include slots, baffles, and or other structures to facilitate guiding and friction-fitting the probe electronic components in place after their insertion into the manually grasped surface. Following 980, method 900 ends.

As provided above, an ultrasound probe having a customized handle is shown and described. In one embodiment, a method of manufacturing an ultrasound probe, may comprise customizing the fit of the ultrasound probe to an operator's hand, including, generating a three-dimensional (3D) model of the operator's hand, digitizing the 3D model of the operator's hand, including obtaining a set of manual attributes, and forming a manually grasped surface of the ultrasound probe based on the digitized 3D model. Furthermore, the manually grasped surface may be coupled to the ultrasound probe. In some examples, obtaining the set of manual attributes of the operator's hand may comprise obtaining one or a combination of a thumb length, a finger length, a palm width, a grasping position, and a probe type. Furthermore, digitizing the 3D model of the operator's hand may comprise mapping a plurality of probe-contact pressure points of the operator's hand into the 3D model, and generating the 3D model of the operator's hand comprises grasping an impressionable material with the hand and forming a physical impression of the operator's hand from the impressionable material, and digitizing the 3D model may comprise 3D scanning the physical impression of the operator's hand to obtain the set of manual attributes. As examples, the impressionable material may comprise one or a combination of clay, foam, plaster, plasticene, gel, and/or other modeling compounds.

Generating the 3D model of the operator's hand and digitizing the 3D model may comprise grasping a probe template with the operator's hand, the probe template including contact sensors, and determining the set of manual attributes based on contact of the operator's hand with the contact sensors. In another example, generating the 3D model of the operator's hand and digitizing the 3D model of the operator's hand may comprise 3D scanning the hand with a 3D scanner. Furthermore, generating the 3D model of the operator's hand may comprise photographing the operator's hand, and digitizing the 3D model comprises generating a point cloud photo model of the hand from one or more photographs of the operator's hand. Further still, the 3D model of the operator's hand may be stored in a database, and digitizing the 3D model may comprise selecting the 3D model of the operator's hand from the database based on the set of manual attributes. In one example, selecting the 3D model of the operator's hand comprises classifying the operator's hand based on the set of manual attributes and selecting the 3D model from a collection of template hand models that matches the classification.

In another embodiment, a method of manufacturing an ultrasound probe, may comprise forming a manually grasped surface of the ultrasound probe corresponding to a model of a grasping hand. In one example, the model may include a set of manual attributes that identify the grasping hand, and the manually grasped surface may comprise a negative surface conforming to a positive surface including the grasping hand. Furthermore, the manually grasped surface may be attached to the ultrasound probe. In some examples, forming the manually grasped surface may comprise one or a combination of 3D printing, molding, and casting the manually grasped surface. Furthermore, forming the manually grasped surface may comprise forming a flexible probe sleeve, and attaching the manually grasped surface to the ultrasound probe may comprise inserting the ultrasound probe into the flexible probe sleeve. Further still, forming the manually grasped surface may comprise forming a hollow rigid housing, and attaching the manually grasped surface to the ultrasound probe may comprise inserting the probe transducer and probe electronics coupled to the probe transducer into the hollow rigid housing. Further still, attaching the manually grasped surface to the ultrasound probe may comprise removably attaching the manually grasped surface to the ultrasound probe.

In another embodiment, an ultrasound probe may comprise a housing, including a manually grasped surface corresponding to a model of a grasping hand, wherein the model includes a set of manual attributes that identify the grasping hand, and the manually grasped surface comprises a negative surface conforming to a positive surface including the grasping hand. The ultrasound probe may further include probe electronics, including an ultrasound probe transducer, positioned inside the housing, and a lens conductively coupled to the probe electronics, positioned at a periphery of the housing, and through which ultrasound radiation is transmitted and received through the housing. In one example, the manually grasped surface may comprise a flexible hollow sleeve removably attached to the housing, an outer surface of the flexibly hollow sleeve comprising the negative surface. Furthermore, an interior surface of the flexible hollow sleeve comprises one or more of a tacky polymer, a coating, and an adhesive. In another example, the manually grasped surface may comprise a rigid hollow surface, and the probe electronics comprise heat dissipation devices positioned adjacent to an interior of the negative surface.

In this way, a technical effect is achieved where ultrasound probes may be designed to be (fully or partially) customizable to the size and shape of a operator's hand, thereby reducing injuries and discomfort due to ergonomic strain and chronic fatigue of the operator's hand and wrist. Furthermore, existing ultrasound probes can be retrofitted with a custom-fit or partially custom-fit ultrasound probe handle, thereby reducing replacement costs. Further still, the custom-fit handles may be removably attached, thereby facilitating repair and reducing replacement costs. Further still, custom-fitting ultrasound probes for each operator can improve hygiene and reduce contamination issues resulting from common ultrasound probes shared amongst several operators. Further still, custom-fitting the ultrasound probe handle to a operator's hand may increase interior free volume within the ultrasound probe, allowing for additional heat dissipation devices to be housed within the ultrasound probe, and thereby reducing degradation and prolonging the useable life of the probe. Further still, custom-fitting ultrasound probes can encourage standardization of hand and wrist posture while grasping ultrasound probes across operators, which can reduce operator to operator variation and increase the reliability of ultrasound imaging.

It is to be understood that the description is intended to be illustrative, and not restrictive. For example, the above-described embodiments (and/or aspects thereof) may be used in combination with each other. In addition, many modifications may be made to adapt a particular situation or material to the teachings of the inventive subject matter without departing from its scope. While the dimensions and types of materials described herein are intended to define the parameters of the inventive subject matter, they are by no means limiting and are exemplary embodiments. Many other embodiments will be apparent to those of ordinary skill in the art upon reviewing the above description. The scope of the inventive subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose several embodiments of the inventive subject matter and also to enable any person of ordinary skill in the art to practice the embodiments of the inventive subject matter, including making and using any devices or systems and performing any incorporated methods. The patentable scope of the inventive subject matter is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

The foregoing description of certain embodiments of the inventive subject matter will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (for example, processors or memories) may be implemented in a single piece of hardware (for example, a general purpose signal processor, microcontroller, random access memory, hard disk, and the like). Similarly, the programs may be stand-alone programs, may be incorporated as subroutines in an operating system, may be functions in an installed software package, and the like. The various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the inventive subject matter are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described systems and methods, without departing from the spirit and scope of the inventive subject matter herein involved, it is intended that all of the subject matter of the above description or shown in the accompanying drawings shall be interpreted merely as examples illustrating the inventive concept herein and shall not be construed as limiting the inventive subject matter.

1. A method of manufacturing an ultrasound probe, comprising:
customizing a fit of the ultrasound probe to an operator's hand, including,
generating a three-dimensional (3D) model of the operator's hand,
digitizing the 3D model of the operator's hand, including obtaining a set of manual attributes, and
forming a manually grasped surface of the ultrasound probe based on the digitized 3D model; and
coupling the manually grasped surface to the ultrasound probe.

2. The method of claim 1, wherein obtaining the set of manual attributes of the operator's hand comprises obtaining one or a combination of a thumb length, a finger length, a palm width, a grasping position, and a probe type.

3. The method of claim 2, wherein digitizing the 3D model of the operator's hand further comprises mapping a plurality of probe-contact pressure points of the operator's hand into the 3D model.

4. The method of claim 3, wherein
generating the 3D model of the operator's hand comprises grasping an impressionable material with the operator's hand and forming a physical impression of the operator's hand from the impressionable material, and
digitizing the 3D model comprises 3D scanning the physical impression of the operator's hand to obtain the set of manual attributes.

5. The method of claim 4, wherein the impressionable material comprises one or a combination of clay, foam, plaster, plasticene, gel, and a modeling compound.

6. The method of claim 3, wherein generating the 3D model of the operator's hand and digitizing the 3D model comprises grasping a probe template with the operator's hand, the probe template including contact sensors, and determining the set of manual attributes based on contact of the operator's hand with the contact sensors.

7. The method of claim 3, wherein generating the 3D model of the operator's hand and digitizing the 3D model of the operator's hand comprises 3D scanning the hand with a 3D scanner.

8. The method of claim 3, wherein generating the 3D model of the operator's hand comprises photographing the operator's hand, and digitizing the 3D model comprises generating a point cloud photo model of the operator's hand from one or more photographs of the operator's hand.

9. The method of claim 3, further comprising storing the 3D model of the operator's hand in a database, wherein digitizing the 3D model comprises selecting the 3D model of the operator's hand from the database based on the set of manual attributes.

10. The method of claim 9, wherein selecting the 3D model of the operator's hand comprises classifying the operator's hand based on the set of manual attributes and selecting the 3D model from a collection of template hand models that matches the classification.

11. A method of manufacturing an ultrasound probe, comprising:
forming a manually grasped surface of the ultrasound probe corresponding to a model of a grasping hand, wherein
the model includes a set of manual attributes that identify the grasping hand, and
the manually grasped surface comprises a negative surface conforming to a positive surface including the grasping hand, and
attaching the manually grasped surface to the ultrasound probe.

12. The method of claim 11, wherein forming the manually grasped surface comprises one or a combination of 3D printing, molding, and casting the manually grasped surface.

13. The method of claim 12, wherein forming the manually grasped surface comprises forming a flexible probe sleeve, and attaching the manually grasped surface to the ultrasound probe comprises inserting the ultrasound probe into the flexible probe sleeve.

14. The method of claim 12, wherein forming the manually grasped surface comprises forming a hollow rigid housing, and attaching the manually grasped surface to the ultrasound probe comprises inserting probe transducer components and probe electronics coupled to the probe transducer into the hollow rigid housing.

15. The method of claim 14, wherein attaching the manually grasped surface to the ultrasound probe comprises removably attaching the manually grasped surface to the ultrasound probe.

16. An ultrasound probe, comprising:
a housing, including a manually grasped surface corresponding to a model of a grasping hand, wherein
the model includes a set of manual attributes that identify the grasping hand, and
the manually grasped surface comprises a negative surface conforming to a positive surface including the grasping hand;
probe electronics, including an ultrasound probe transducer, positioned inside the housing; and
a lens conductively coupled to the probe electronics, positioned at a periphery of the housing, and through which ultrasound radiation is transmitted and received through the housing.

17. The ultrasound probe of claim 16, wherein the manually grasped surface comprises a flexible hollow sleeve removably attached to the housing, an outer surface of the flexibly hollow sleeve comprising the negative surface.

18. The ultrasound probe of claim 17, wherein an interior surface of the flexible hollow sleeve comprises one or more of a tacky polymer, a coating, and an adhesive.

19. The ultrasound probe of claim 16, wherein the manually grasped surface comprises a rigid hollow surface.

20. The ultrasound probe of claim 16, wherein the probe electronics comprise heat dissipation devices positioned adjacent to an interior of the negative surface.
